(19) United States
(12) Patent Application Publication
(10) Pub. No.: US 2007/0156168 A1
(43) Pub. Date: Jul. 5, 2007

(54) Polymer marker and retention bands

(75) Inventors: Albert Dunfee, Byfield, MA (US); Barbara DeVaux, Haverhill, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/322,154

(22) Filed: Dec. 29, 2005

(51) Int. Cl. A61M 29/00

(52) U.S. Cl. 606/200

(57) ABSTRACT

A filtering device having an embolic filter for collecting debris in a body lumen during an intravascular procedure includes a filter proximal neck portion attached to a proximal shaft and a filter distal neck portion attached to a distal support shaft. The proximal shaft is slidably disposed about a core wire and the distal support shaft is attached to the core wire. Relative longitudinal movement between the proximal shaft and the core wire moves the filter ends closer together to expand the filter or moves the filter ends farther apart to collapse the filter. The proximal and distal neck portions comprise wires of the braided filter with interstices there between. A radiopaque polymer marker band is disposed about the proximal neck portion of the filter and is melt bonded to the proximal shaft through the interstices between the wires, thereby securing the proximal neck portion. A second radiopaque polymer marker band is disposed about the distal neck portion of the filter and is melt bonded to the distal support shaft through the interstices between the wires, thereby securing the distal neck portion. The radiopaque polymer marker bands comprise a radiopaque polymer or a radiolucent polymer doped with a radiopaque material.

### THE INVENTION

The invention relates generally to intraluminal distal protection devices for capturing particulate in the vessels of a patient. More particularly, the invention relates to a filter for capturing emboli in a blood vessel during an interventional vascular procedure.

### GROUND OF THE INVENTION

Catheters have long been used for the treatment of diseases of the cardiovascular system, such as treatment or removal of stenosis. For example, in a percutaneous transluminal coronary angioplasty (PTCA) procedure, a catheter is used to insert a balloon into a patient's cardiovascular system, position the balloon at a desired treatment location, inflate the balloon, and remove the balloon from the patient. Another example is the placement of a prosthetic stent that is placed in the body on a permanent or semi-permanent basis to support weakened or diseased vascular walls to avoid closure or rupture thereof.

These non-surgical interventional procedures often avoid the necessity of major surgical operations. However, one common problem associated with these procedures is the potential release into the bloodstream of embolic debris that can occlude distal vasculature and cause significant health problems to the patient. For example, during deployment of a stent, it is possible for the metal struts of the stent to cut into the stenosis and shear off pieces of plaque which become embolic debris that can travel downstream and lodge somewhere in the patient's vascular system. Further, pieces of plaque material can sometimes dislodge from the stenosis during a balloon angioplasty procedure and become released into the bloodstream.

Medical devices have been developed to attempt to deal with the problem created when debris or fragments enter the circulatory system during vessel treatment. One technique includes the placement of a filter or trap downstream from the treatment site to capture embolic debris before it reaches the smaller blood vessels downstream. The placement of a filter in the patient's vasculature during treatment of the vascular lesion can collect embolic debris in the bloodstream.

It is known to attach an expandable filter to a distal end of a guidewire or guidewire-like member that allows the filtering device to be placed in the patient's vasculature. The guidewire allows the physician to steer the filter to a downstream location from the area of treatment. Once the guidewire is in proper position in the vasculature, the embolic filter can be deployed to capture embolic debris. Some embolic filtering devices utilize a restraining sheath to maintain the expandable filter in its collapsed configuration. Once the proximal end of the restraining sheath is retracted by the physician, the expandable filter will transform into its fully expanded configuration. The restraining sheath can then be removed from the guidewire allowing the guidewire to be used by the physician to deliver interventional devices, such as a balloon angioplasty catheter or a stent delivery catheter, into the area of treatment. After the interventional procedure is completed, a recovery sheath can be delivered over the guidewire using over-the-wire techniques to collapse the expanded filter (with the trapped embolic debris) for removal from the patient's vasculature. Both the delivery sheath and recovery sheath should be relatively flexible to track over the guide wire and to avoid straightening the body vessel once in place.

Another distal protection device known in the art includes a filter mounted on a distal portion of a hollow guidewire or tube. A moveable core wire is used to open and close the filter. The filter is coupled at a proximal end to the tube and at a distal end to the core wire. Pulling on the core wire while pushing on the tube draws the ends of the filter toward each other, causing the filter framework between the ends to expand outward into contact with the vessel wall. Filter mesh material is mounted to the filter framework. To collapse the filter, the procedure is reversed, i.e., pulling the tube proximally while pushing the core wire distally to force the filter ends apart. A sheath catheter may be used as a retrieval catheter at the end of the interventional procedure to reduce the profile of the “push-pull” filter, as due to the embolic particles collected, the filter may still be in a somewhat expanded state. The retrieval catheter may be used to further collapse the filter and smooth the profile thereof, so that the filter guidewire may pass through the treatment area without disturbing any stents or otherwise interfering with the treated vessel.

However, regardless of how a distal protection filter is expanded during a procedure, i.e., sheath delivered or by use of a push-pull mechanism, a crossing profile of the collapsed filter is to be at a minimum to reduce interference between the filter and other interventional devices or in-placed stents. As well, a compact filter profile is beneficial in crossing severely narrowed areas of vascular stenosis.

In existing devices, the main factors contributing to the crossing profile are the wire diameter of the filter material and the marker band outer diameter. The filter material is generally inserted at each end underneath marker bands and then glued in place. Adequate space between marker bands and the filter material is required for the glue to wick around the filter material and under the marker bands, increasing the thickness in this area. Thus, what is needed is a reduced diameter method to secure the filter material to an underlying surface with a marker band surrounding the filter material.

### BRIEF SUMMARY OF THE INVENTION

The present invention is a filtering device having a braided embolic filter for collecting debris in a body lumen during an intravascular procedure. A filter proximal neck portion is attached to a proximal shaft and a filter distal neck is attached to a distal support shaft. The proximal shaft is slidably disposed about a core wire and the distal support shaft is attached to the core wire. Relative longitudinal movement between the proximal shaft and the core wire moves the filter ends closer together to expand the filter or moves the filter ends farther apart to collapse the filter.

The proximal and distal neck portions comprise wires of the braided filter with interstices (spaces) there between. A radiopaque polymer marker band is disposed about the proximal neck portion of the filter and is melt bonded to the proximal shaft through the interstices between the wires, thereby securing the proximal neck portion to the proximal shaft without substantially adding wall thickness, or overall diameter to the neck portion. A second radiopaque polymer marker band is disposed about the distal neck portion of the filter and is melt bonded to the distal support shaft through the interstices between the wires, thereby securing the distal neck portion without substantially adding wall thickness, or overall diameter to the neck portion.

The radiopaque polymer marker bands comprise a radiopaque polymer or a radiolucent polymer doped with a radiopaque material. The marker bands are melt bonded to the underlying surface, preferably with the use of heat shrink tubing and a heat source, wherein the heat simultaneously causes the marker band to melt and the heat shrink tubing to exert a compressive force on the underlying molten marker band material. The heat shrink tubing is subsequently removed.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 is an illustration of a filter system in accordance with an embodiment of the present invention deployed within a blood vessel.

FIG. 2 is an illustration of a filter system in accordance with an embodiment of the present invention deployed within the coronary arterial anatomy.

FIG. 3 is a side view of an embodiment of a distal protection device in accordance with the present invention.

FIG. 4 is an enlarged view of portion A of the embodiment shown in FIG. 3, shown before the marker band is melt bonded to the underlying surface.

FIG. 5 is an enlarged view of portion B of the embodiment shown in FIG. 3, shown before the marker band is melt bonded to the underlying surface.

FIG. 6 is a side view of the distal neck portion of the filter of FIG. 3, shown with the marker band (portions removed for clarity) melt bonded to the underlying surface in accordance with the invention.

FIG. 7 is a transverse cross section taken along line C-C of FIG. 6.

### DETAILED DESCRIPTION OF THE INVENTION

Specific embodiments of the present invention are now described with reference to the figures, where like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

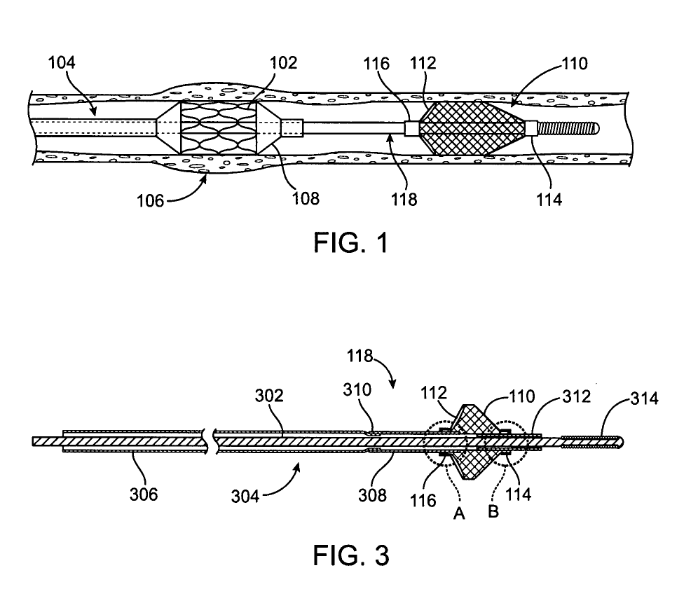

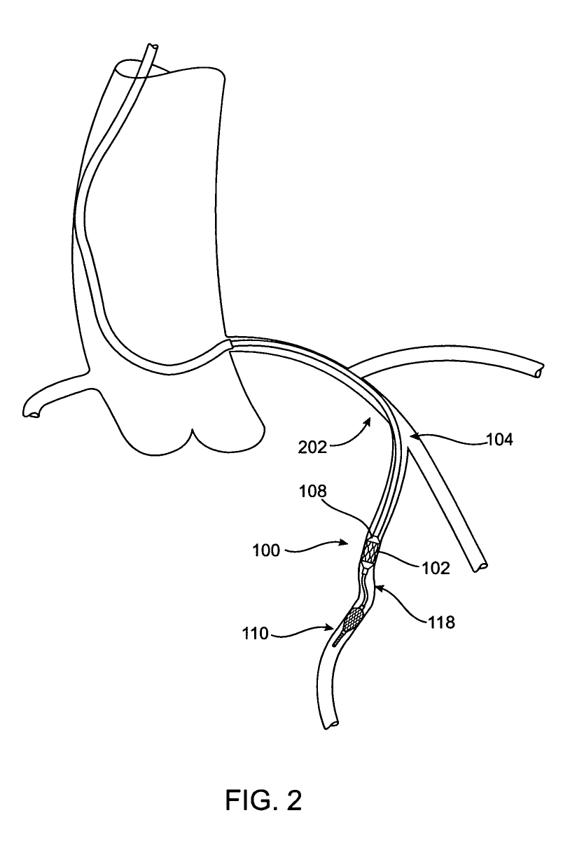

The present invention is a guidewire apparatus for use in minimally invasive procedures. While the following description of the invention relates to vascular interventions, it is to be understood that the invention is applicable to other procedures where the practitioner desires to capture embolic material that may be dislodged during the procedure. Intravascular procedures such as PTCA or stent deployment are often preferable to more invasive surgical techniques in the treatment of vascular narrowings, called stenoses or lesions. With reference to FIGS. 1 and 2, deployment of balloon expandable stent 102 is accomplished by threading catheter 104 through the vascular system of the patient until stent 102 is located within a stenosis at predetermined treatment site 106. Once positioned, balloon 108 of catheter 104 is inflated to expand stent 102 against the vascular wall to maintain the opening. Stent deployment can be performed following treatments such as angioplasty, or during initial balloon dilation of the treatment site, which is referred to as primary stenting.

Catheter 104 is typically guided to treatment site 106 by a guidewire 118. In cases where the target stenosis is located in tortuous vessels that are remote from the vascular access point, such as coronary arteries 202 shown in FIG. 2, a steerable guidewire is commonly used. According to the present invention, a guidewire apparatus generally guides catheter 104 to treatment site 106 and includes a distally disposed protection element to collect embolic debris that may be generated during the procedure. Various embodiments of the invention will be described as either filter guidewires or occluder guidewires. However, it is to be understood that filters and occluders are interchangeable types of protection elements among the inventive structures disclosed. The invention is directed to embolic protection elements wherein relative movement of the ends of the protection element either causes or accompanies transformation of the element between a collapsed configuration and an expanded or deployed configuration. Such transformation may be impelled by external mechanical means or by self-shaping memory (either self-expanding or self-collapsing) within the protection element itself. The protection element may be self-expanding, meaning that it has a mechanical memory to return to the expanded or deployed configuration. Such mechanical memory can be imparted to the metal comprising the element by thermal treatment to achieve a spring temper in stainless steel, for example, or to set a shape memory in a susceptible metal alloy such as a nickel-titanium (nitinol) alloy.

Filter guidewires in accordance with the invention include distally disposed filter 110, which may comprise a tube formed by braided filaments that define pores and have at least one proximally-facing inlet opening 112 that is substantially larger than the pores. Radiopaque markers 114, 116 are disposed about the distal filter end and the proximal filter end to aid in fluoroscopic observation of filter 110 during manipulation thereof. Optionally, at least one of the filaments of braided filter 110 may be a wire having enhanced radiopacity compared to conventional non-radiopaque wires suitable for braided filter 110.

The present invention is directed to a distal protection device, in particular, a filter 110 mounted on filter guidewire 118, which has a reduced profile in its collapsed configuration or state. FIG. 3 illustrates the filter and guidewire arrangement according to an embodiment of the present invention. Filter guidewire 118 includes an elongate core, such as core wire 302, and flexible tubular tip member 314, such as a coil spring fixed around the distal end of core wire 302. Thin wires made from stainless steel and/or one of various alloys of platinum are commonly used to make coil springs for such use in guidewires. Core wire 302 can be made from shape memory metal such as nitinol, or a stainless steel wire, and is typically tapered at its distal end. For treating small caliber vessels such as coronary arteries, core wire 302 may measure about 0.15 mm (0.006 inch) in diameter.

In filter guidewire 118, hollow shaft 304 is movably disposed around core wire 302, and includes relatively stiff proximal portion 306 and relatively flexible distal portion 308. Proximal portion 306 may be made from thin walled stainless steel tubing, usually referred to as hypo tubing, although other metals, such as nitinol, can be used. Various metals or polymers can be used to make relatively flexible distal portion 308, although in the present invention a polymer material is preferred for compatibility with marker band 116, as described in more detail below. Distal portion 308 can be made of materials including, but not limited to, polyamide, polyimide, polyurethane, polyethylene, or polyethylene block amide copolymer. The length of distal portion 308 may be selected as appropriate for the intended use of the filter guidewire. In one example, portion 308 may be designed and intended to be flexible enough to negotiate tortuous coronary arteries, in which case the length of portion 308 may be 15-35 cm (5.9-13.8 inches), or at least approximately 25 cm (9.8 inches). In comparison to treatment of coronary vessels, adaptations of the invention for treatment of renal arteries may require a relatively shorter flexible portion 308, and neurovascular versions intended for approaching vessels in the head and neck may require a relatively longer flexible portion 308.

When filter guidewire 118 is designed for use in small vessels, shaft 304 may have an outer diameter of about 0.36 mm (0.014 inch). The general uniformity of the outer diameter may be maintained by connecting proximal portion 306 and distal portion 308 with a lap joint 310. Lap joint 310 may use any suitable biocompatible adhesive such as ultraviolet (UV) light curable adhesives, thermally curable adhesives or so-called “instant” cyanoacrylate adhesives from Dymax Corporation, Torrington, Conn., U.S.A or Loctite Corporation, Rocky Hill, Conn., U.S.A. Lap joint 310 can be formed by any conventional method such as reducing the wall thickness of proximal portion 306 in the region of joint 310, or by forming a step-down in diameter at this location with negligible change in wall thickness, as by swaging. Other joints, such as butt joints may be used to join proximal portion 306 and distal portion 308.

Expandable tubular filter 110 is positioned generally concentrically with core wire 302, and is sized such that when it is fully deployed, as shown in FIGS. 1 and 2, the outer perimeter of filter 110 will contact the inner surface of the vessel wall. The surface contact is maintained around the entire vessel lumen to prevent any emboli from slipping past filter 110.

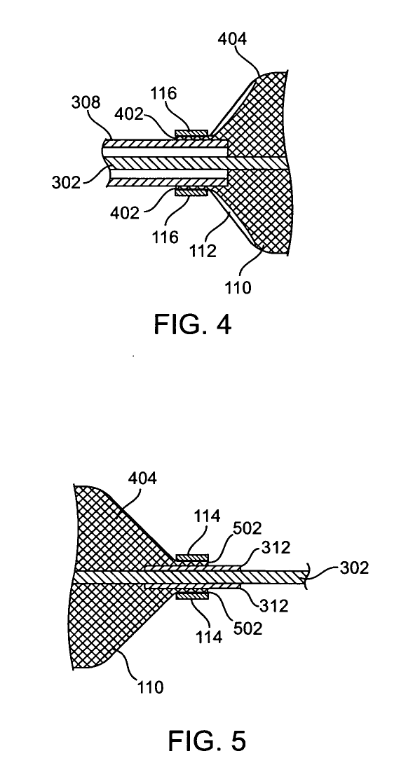

In the embodiment of FIG. 3, filter 110 is a braided filter comprised of a plurality of wires or filaments 404 (shown in FIG. 4) that are woven together to form the tubular braided filter. In an embodiment of the present invention, braiding wires or filaments are preferably made from stainless steel, a shape memory material, such as nitinol, or a nickel-based super alloy. Wires 404 may have a round cross-section, as shown, or may have a square, flat or ribbon, oval or other cross-sectional shape. Wire cross-sections that are not square or round are typically braided into a tube with their thinnest transverse dimension oriented with the radius of the tube, e.g. so as to provide a low radial thickness of the braided tube, whether such thickness is measured at intersections where wires 404 cross over each other, or at braid locations between the intersections. In another embodiment, the distal filter portion may be formed from a suitable mesh or porous material that collects embolic debris while permitting fluid to flow there through, such as blood flow sufficient for perfusion of body tissues. Such mesh filters and braided filters are disclosed in U.S. Pat. No. 6,346,116 that is incorporated by reference herein in its entirety.

Filter 110 includes a proximal neck portion 402 that is attached to distal portion 308 of shaft 304. Proximal marker band 116 is disposed around proximal neck portion 402 of filter 110 and attaches proximal neck portion 402 to distal portion 308, as shown in FIG. 4 before marker band 116 has been melt bonded to encapsulate proximal neck portion 402.

In the current example, a distal support shaft 312 is attached to a distal portion of core wire 302. Distal support shaft 312 is preferably a polymer material, such as polyamide, polyimide, polyurethane, polyethylene, or polyethylene block amide copolymer. However, distal support shaft 312 can be made of any material that is compatible with distal marker band 114 such that distal marker band 114 can be melt bonded to distal support shaft 312. A distal neck portion 502 of filter 110 is attached via distal marker band 114 to distal support shaft 312. Distal support shaft 312 can provide a rotatable connection between distal neck portion 502 and core wire 302. Distal support shaft 312 can also provide an intermediate surface for melt bonding distal neck portion 502 about core wire 302 where the surface of core wire 302 itself is incompatible for melt bonding with marker band 114. Distal marker band 114 is disposed around distal neck portion 502 of filter 110 and attaches distal neck portion 502 to distal support shaft 312, as shown in FIG. 5 before marker band 114 has been melt bonded to encapsulate distal neck portion 502. In an alternative embodiment, distal marker band 114 can be melt bonded through distal neck portion 502 directly to core wire 302, eliminating distal support shaft 312 and the possibly disadvantageous additional thickness thereof.

Filter 110 is deployed by advancing, or pushing shaft 304 relative to core wire 302 such that filter distal and proximal ends 502, 402 are drawn toward each other, forcing the middle, or central section of filter 110 to expand radially. Filter 110 is collapsed by withdrawing, or pulling shaft 304 relative to core wire 302 such that filter distal and proximal ends 502, 402 are drawn apart from each other, forcing the middle, or central section of filter 110 to contract radially. Proximal and distal stops (not shown) may be utilized to limit the relative longitudinal movement between the core wire 302 and the proximal shaft 304.

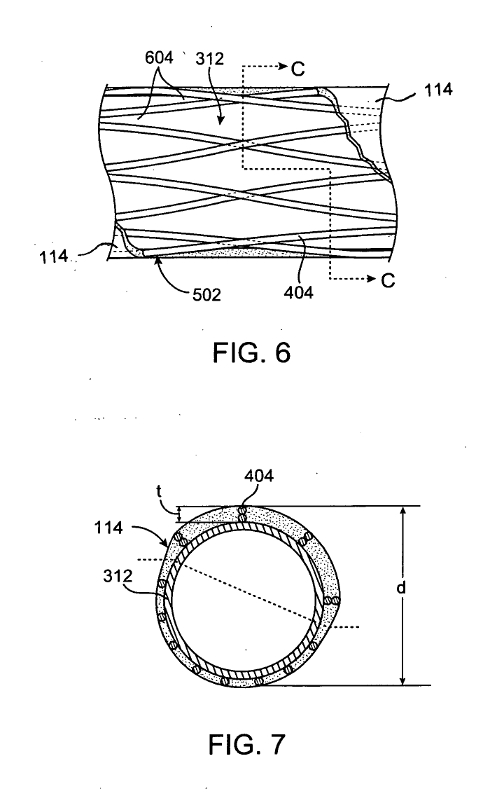

In the present invention, distal and proximal marker bands 114, 116 serve the dual purpose of aiding in fluoroscopic observation of filter 110 during manipulation thereof and attaching the distal and proximal neck portions 502, 402 to a respective underlying surface, which, in the embodiment of FIG. 3, comprise distal support shaft 312 and distal portion 308 of shaft 304, respectively. As noted above, filter 110 comprises a plurality of wires or filaments 404 that are woven together. The wires or filaments come together at the distal and proximal neck portions 502, 402, with interstices between the crossing wires. FIG. 6 shows a side view of this arrangement at distal neck portion 502, showing distal support shaft 312 and the wires 404 of distal neck portion 502, with interstices 604 between the wires. Distal marker band 114 is placed over wires 404 and is melt bonded to distal support shaft 312 through interstices 604, thereby encapsulating or capturing wires 404 and securing distal neck portion 502 to distal support shaft 312. FIG. 6 shows distal marker band 114 after melt bonding. However, portions of marker band 114 have been removed for clarity. Similarly, proximal marker band 116 is melt bonded to distal portion 308 of shaft 304 through the interstices between the wires of proximal neck 402.

The marker bands are melt bonded to the underlying surface, preferably with the use of heat shrink tubing (not shown) and a heat source (hot air, radiant heater, laser, etc.) wherein the heat simultaneously causes the marker band to melt and the heat shrink tubing to exert a compressive force on the underlying molten material. The heat shrink tubing is used as a disposable tool that is removed from the assembly after the melt bonded marker band has cooled. The use of heat shrink tubing as a removable tool in a method of simultaneously compressing and melt bonding a thermoplastic tube is known by those of skill in the art of medical catheters and guidewires. An exemplary temperature range for melting a marker band and simultaneously shrinking a heat shrink tube is about 171-210° C. Melt bonding the marker bands onto distal portion 308 and distal support shaft 312 provides the added benefit of significantly reducing the diameter or profile of the bonded neck portions 402, 502. The edges of the marker bands may also be slightly tapered to reduce the likelihood of catching an edge and either damaging the marker bands or the distal protection device during assembly or handling of the distal protection device.

FIG. 7 shows a transverse cross-sectional view taken along line C-C of FIG. 6 after distal marker band 114 is melt bonded to distal support shaft 302 through interstices 604. Line C-C is jogged longitudinally so that FIG. 7 illustrates, in one view, sections of neck 502 where braided wires 404 cross and also sections of neck 502 where braided wires 404 do not cross. By using heat shrink tubing to melt bond distal marker band 114 to distal support shaft 312, the compressive force of the heat shrink tubing forces distal marker band 114 into interstices 604. The heat shrink tubing is sufficiently hard during heat shrinking so that its inner diameter will substantially stop shrinking when the tubing contacts wires 404. Distal marker band 114 will thus be compressed into interstices 604 such that distal marker band 114 does not add substantially to the overall diameter d of filter guidewire 118 at distal neck portion 502. Thus, the overall diameter d is substantially the same with the distal marker band 114 melt bonded in place as it would be without distal marker band 114.

As shown in FIG. 7, the overall diameter d is comprised of the sum of the diameter of distal support tube 312 and twice the thickness t of distal neck 502. Thickness t of distal neck 502 may equal the radial thickness of either one or two wires 404, depending on where thickness t is measured. At locations where wires 404 intersect, overall diameter d is comprised of the sum of the diameter of distal support tube 312 and four (4) times the radial thickness or diameter of wires 404. At locations where wires 404 do not intersect, but lie side-by-side, overall diameter d is comprised of the sum of the diameter of distal support tube 312 and two (2) times the radial thickness or diameter of wires 404. The melt bonded distal marker band 114 fits within the thickness t of the distal neck 502, thereby not adding to the overall diameter d of the distal neck portion of the device.

Marker bands 114, 116 may be formed of a radiolucent polymer doped with a radiopaque material. For example, the polymer can be any of a variety of suitable polymers, including, but not limited to polyethylene block amide copolymer, polyethylene, linear low density polyethylene, alpha olefin copolymers, polyester, polyamide, thermoplastic polyetherurethane elastomers, thermoplastic polyester elastomers, olefin-derived copolymers, natural and synthetic thermoplastic rubbers like silicone (polydimethylsiloxane/urea copolymer) and isoprene and specialty polymers like ethylene vinyl acetate (EVA) and ionomers, etc. as well as alloys thereof. The polymer must be compatible with the material of the underlying surface, such as distal support shaft 312 and distal section 308 of shaft 304, respectively. The material preferably comprises a low durometer polymer in order to render the marker sufficiently flexible so as not to impair the flexibility of the underlying medical device component to which the finished marker is to be attached. The polymer must also impart sufficient strength and ductility to the marker compound so as to facilitate its extrusion and forming into a marker, its subsequent handling and attachment to a medical device and preservation of the marker's integrity as the distal protection device is flexed and manipulated during use. The material for proximal and distal marker bands can be different and may be selected, in conjunction with the material of the underlying surface, to impart a different flexibility on the particular section of the distal protection device. For example, it is generally desirable to have the more distal sections of the device be more flexible than the proximal portions.

The radiopaque material used to dope the polymer can be any of a number of different metals that are well known to have suitable x-ray attenuation coefficients, and can be used in pure or alloyed form. Commonly used metals include, but are not limited to, platinum, gold, iridium, palladium, rhenium, rhodium, tungsten, tantalum, silver, and tin. Bismuth subcarbonate and barium sulfate are also suitable radiopaque doping agents. Materials for radiopaque polymeric marker bands 114, 116 of the present invention can be those described in U.S. Pat. No. 6,540,721 and U.S. Published Patent Application Publication No. 2005/0064223, both of which are incorporated by reference herein in their entirety.

Although a particular embodiment of a filter device has been shown and described, it will be understood by persons skilled in the relevant art that there are various filter devices that can utilize radiopaque polymer marker bands securing the wires or filaments of a filter to an underlying surface. Such radiopaque polymer marker bands can be utilized, for example, in the various embodiments described in U.S. Pat. Nos. 6,706,055, 3,346,116, 6,818,006, and 6,866,677, all of which are incorporated by reference herein in their entirety.

While various embodiments of the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A filtering device for collecting debris in a body lumen, comprising:
a core wire;
a proximal shaft disposed about the core wire , the proximal shaft having a distal portion;
a distal support shaft disposed about the core wire distally of the proximal shaft;
a filter having a proximal neck disposed about the distal portion of the proximal shaft, a distal neck disposed about the distal support shaft, and an opening for receiving debris in a proximal portion of the filter, wherein relative longitudinal movement between the proximal shaft and the distal support shaft accompanies transformation of the filter between a collapsed configuration and a deployed configuration; and
a radiopaque polymer marker band disposed about one of the proximal or distal necks, wherein the radiopaque polymer marker band is melt bonded to the distal portion or the distal support shaft, thereby securing the proximal or distal neck.

2. The filtering device of claim 1, further comprising a second radiopaque polymer marker band, wherein the second radiopaque polymer marker bond is disposed about the other of the proximal or distal necks and is melt bonded to the distal portion or the distal support shaft, thereby securing the proximal or distal neck.

3. The filtering device of claim 1, wherein the radiopaque polymer marker band comprises a polymer material doped with a radiopaque material.

4. The filtering device of claim 3, wherein the polymer material is selected from the group consisting of polyethylene block amide copolymer, polyethylene, linear low density polyethylene, alpha olefin copolymer, polyester, polyamide, thermoplastic polyetherurethane elastomers, thermoplastic polyester elastomers, olefin-derived copolymers, natural thermoplastic rubber, synthetic thermoplastic rubber, silicone rubber, polydimethylsiloxane/urea copolymer, isoprene, ethylene vinyl acetate, ionomers and alloys thereof.

5. The filtering device of claim 4, wherein the radiopaque material is selected from the group consisting of bismuth subcarbonate, barium sulfate, platinum, gold, iridium, palladium, rhenium, rhodium, tungsten, tantalum, silver, and tin.

6. The filtering device of claim 1, wherein the distal support shaft is secured to the core wire.

7. The filtering device of claim 6, wherein the proximal shaft is slidable relative to the core wire.

8. The filtering device of claim 1, wherein the distal portion of the proximal shaft or the distal support shaft comprises a material selected from the group consisting of polyamide, polyimide, polyurethane, polyethylene and polyethylene block amide copolymer.

9. The filtering device of claim 1, wherein the filter comprises braided wires, a thickness of the proximal or distal necks comprises not more than twice the radial thickness of the wires, and the radiopaque polymer marker band is disposed within the thickness after being melt bonded to the distal portion or the distal support shaft, such that a combined thickness of the proximal or distal neck and the radiopaque polymer marker band melt bonded in place is substantially the same as the thickness of the proximal or distal neck.

10. A method of securing a filter to a distal protection device comprising the steps of:
disposing the filter about an elongate core, wherein the filter includes a neck portion disposed about an underlying surface, the neck portion comprising wires;
disposing a radiopaque polymer marker band about the neck portion; and
melt bonding the radiopaque polymer marker band to the underlying surface such that the neck portion is secured to the underlying surface.

11. The method of claim 10, wherein the radiopaque polymer markers band comprises a polymer material doped with a radiopaque material.

12. The method of claim 11, wherein the polymer material is selected from the group consisting of polyethylene block amide copolymer, polyethylene, linear low density polyethylene, alpha olefin copolymer, polyester, polyamide, thermoplastic polyetherurethane elastomers, thermoplastic polyester elastomers, olefin-derived copolymers, natural thermoplastic rubber, synthetic thermoplastic rubber, silicone rubber, polydimethylsiloxane/urea copolymer, isoprene, ethylene vinyl acetate, ionomers and alloys thereof.

13. The method of claim 12, wherein the radiopaque material is selected from the group consisting of bismuth subcarbonate, barium sulfate, platinum, gold, iridium, palladium, rhenium, rhodium, tungsten, tantalum, silver, and tin.

14. The method of claim 10, wherein the underlying surface comprises a distal portion of a proximal shaft and is slidable relative to the core.

15. The method of claim 14, wherein the distal portion comprises a material selected from the group consisting of polyamide, polyimide, polyurethane, polyethylene and polyethylene block amide copolymer.

16. The method of claim 10, wherein the underlying surface comprises a distal support shaft secured to the core.

17. The method of claim 16, wherein the distal support shaft comprises a material selected from the group consisting of polyamide, polyimide, polyurethane, polyethylene, and polyethylene block amide copolymer.

18. The method of claim 10, wherein the filter comprises braided wires, wherein a thickness of the neck comprises not more than twice the radial thickness of the braided wires, and wherein the step of melt bonding the radiopaque polymer marker band to the underlying surface forces the radiopaque polymer marker band through interstices between the braided wires such that the radiopaque polymer marker bands are disposed within the thickness of the neck such that a combined thickness of the neck and the radiopaque polymer marker band is substantially the same as the thickness of the neck.

19. A medical device comprising:
an elongate core;
a device comprising wires or filaments disposed about the core; and
a radiopaque polymer marker band disposed about the device, wherein the radiopaque polymer marker band is melt bonded to the core and secures the device to the core.

20. The medical device of claim 19, wherein the radiopaque polymer marker band comprises a thermoplastic polymer material doped with a radiopaque material.

21. The medical device of claim 20, wherein the polymer material is selected from the group consisting of polyethylene block amide copolymer, polyethylene, linear low density polyethylene, alpha olefin copolymer, polyester, polyamide, thermoplastic polyetherurethane elastomers, thermoplastic polyester elastomers, olefin-derived copolymers, natural thermoplastic rubber, synthetic thermoplastic rubber, silicone rubber, polydimethylsiloxane/urea copolymer, isoprene, ethylene vinyl acetate, ionomers and alloys thereof.

22. The medical device of claim 21, wherein the radiopaque material is selected from the group consisting of bismuth subcarbonate, barium sulfate, platinum, gold, iridium, palladium, rhenium, rhodium, tungsten, tantalum, silver, and tin.

23. The medical device of claim 19, wherein the device disposed about the core is a braided filter.

24. The medical device of claim 19, wherein the tube comprises a material selected from the group consisting of polyamide, polyimide, polyurethane, polyethylene and polyethylene block amide copolymer.

25. The medical device of claim 19, wherein the wires or filaments overlap each other to define a thickness not more than twice the diameter of the wires or filaments, wherein the radiopaque polymer marker band is disposed within the thickness after being melt bonded to the core, such that a combined thickness of the wires or filaments and the radiopaque polymer marker band melt bonded in place is substantially the same as the thickness.
